(19) United States
(12) Patent Application Publication
(10) Pub. No.: US 2009/0326330 A1
(43) Pub. Date: Dec. 31, 2009

(54) APPARATUS FOR INSERTING A SURGICAL DEVICE AT LEAST PARTIALLY THROUGH A WOUND OPENING

(76) Inventors: Frank Bonadio, County Wicklow (IE); John Butler, County Dublin (IE); Trevor Vaugh, County Offaly (IE); Shane Joseph MacNally, County Wickow (IE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/374,941

(22) PCT Filed: Jul. 26, 2007
(86) PCT No.: PCT/IE07/00073

§ 371 Date: May 26, 2009

Related U.S. Application Data
(60) Provisional application No. 60/833,478, filed on Jul. 27, 2006.
(60) Provisional application No. 60/841,242, filed on Aug. 31, 2006.

(51) Int. Cl. A61B 1/32

(52) U.S. Cl. 600/201

(57) ABSTRACT

An apparatus (1) for inserting a wound retractor device (2) at least partially through a wound opening. The apparatus (1) comprises a conveying device (4) and a holder element (5). The conveying device (4) is insertable at least partially through the wound opening to convey the would retractor device (2) at least partially through the wound opening. The holder element (5) holds part of the wound retractor device (2) in a low-profile configuration. The holder element (5) is slidable relative to the conveying device (4) between a holding configuration in which part of the wound retractor device (2) is held in the low-profile configuration, and a release configuration in which the wound retractor device (2) is released. As the conveying device (4) is inserted through the wound opening, the holder element (5) engages with the external surface of the tissue. In this manner the holder element (5) is moved from the holding configuration to the release configuration.

### INTRODUCTION

This invention relates to an apparatus for inserting a surgical device at least partially through a wound opening, to a method of inserting the surgical device at least partially through the wound opening, and to an opening device to create a wound opening.

### STATEMENTS OF INVENTION

According to the invention there is provided an apparatus for inserting a surgical device at least partially through a wound opening, the apparatus comprising:—

a conveying device insertable at least partially through a wound opening to convey a surgical device at least partially through the wound opening; and
a holder element for holding at least part of the surgical device in a low-profile configuration.

It has been found that the apparatus according to the invention results in particularly low profile insertion of the surgical device. The holder element facilitates ease of insertion of the surgical device, while minimising trauma to the sides of the wound opening. In addition the process of insertion of the surgical device using the apparatus according to the invention is a particularly simple process with a minimum number of stages required by the user.

In one embodiment of the invention the holder element is configured to be disassembled from the conveying device. The holder element may be configured to be disassembled from the conveying device after conveying of a surgical device at least partially through a wound opening. The holder element may be configured to be disassembled from a surgical device. The holder element may comprise a circumferential discontinuity. The circumferential discontinuity facilitates disassembly of the holder element from the surgical device.

In one embodiment of the invention the holder element is movable relative to the conveying device between a holding configuration in which at least part of a surgical device is held in a low-profile configuration, and a release configuration in which the surgical device is released. The holder element may be slidable relative to the conveying device between the holding configuration and the release configuration. The holder element may be slidable relative to the conveying device along the conveying device. The holder element may be engagable with an external surface of tissue upon insertion of the conveying device at least partially through a wound opening to move the holder element from the holding configuration to a release configuration. The holder element may be movable radially relative to the conveying device between the holding configuration and the release configuration. The holder element may be configured to be arranged concentrically around the conveying device at least in the holding configuration. The holder element may be removable from being concentrically around the conveying device.

The holder element may be configured to be arranged radially offset from the conveying device at least in the holding configuration.

In one embodiment the holder element is movable relative to the conveying device between a storage configuration in which at least part of a surgical device is released, and the holding configuration. Because the surgical device is released in the storage configuration, this enables the surgical device to be stored for relatively long periods of time without damaging or degrading the surgical device. The holder element may be slidable relative to the conveying device between the storage configuration and the holding configuration. The holder element may be configured to be arranged concentrically around at least part of a surgical device in the storage configuration. The holder element may comprise one or more openings through which part of a surgical device may extend in the storage configuration.

The holder element may be configured to extend radially outwardly from the conveying device. In one case the holder element comprises an annular flange element. In another case the holder element comprises a tubular element.

In one embodiment the holder element is configured to assist collapse of at least part of a surgical device from a release configuration to a low-profile configuration. At least part of the holder element may comprise a funnel part. At least part of the holder element may be tapered. At least part of the holder element may taper radially inwardly distally.

The holder element may be non-circular in cross-section. The holder element may be elliptical in cross-section.

The holder element may be configured to be attached to a surgical device.

In one case the conveying device comprises a retaining element to retain a surgical device in engagement with the conveying device, and a passageway to facilitate location of the surgical device in engagement with the conveying device, the retaining element being configured to selectively obstruct the full width of the passageway. The proximal end of the retaining element may be located proximally of the proximal side of the passageway. The distal end of the retaining element may be located distally of the distal side of the passageway. The retaining element may overlap the passageway.

In another embodiment the conveying device comprises a hook element to engage a surgical device. The hook element may be non-symmetrical in cross-section. The hook element may be non-circular in cross-section.

In a further embodiment the apparatus comprises an opening device to create a wound opening. The opening device may be configured to create a wound opening by forcing tissue apart. The opening device may be mounted to the conveying device. The opening device may be non-symmetrical in cross-section. The major axis of the opening device may be substantially aligned with the major axis of the hook element. By aligning the major axes, this arrangement minimises the profile of the apparatus and the surgical device, and minimises the trauma on the tissue surrounding the wound.

In another aspect of the invention there is provided a surgical assembly comprising:—

a surgical device; and
an apparatus of the invention for inserting the surgical device at least partially through a wound opening.

In one embodiment of the invention the surgical device comprises a wound retractor device. The wound retractor device may comprise a distal member for location internally of a wound opening to anchor the wound retractor device in position retracting the wound opening.

The assembly may comprise a pack for the surgical device and the apparatus. The holder element may be movable relative to the conveying device in the pack between the storage configuration and the holding configuration.

In another aspect the invention provides an opening device to create a wound opening, the size of at least part of the opening device being adjustable to vary the size of the wound opening created.

In one embodiment of the invention the lateral dimension of the opening device is adjustable. The width of the opening device may be adjustable.

In one embodiment of the invention the opening device comprises a distal head, the size of the distal head being adjustable.

In one case the opening device is configured to create a wound opening by forcing tissue apart. At least part of the opening device may be non-symmetrical in cross-section. The size of at least part of the opening device may be adjustable in a direction parallel to the major axis of the opening device.

In another embodiment the device comprises one or more wing elements. The wing element may be movable relative to a main body portion of the device between a narrowed configuration and a widened configuration.

In another case the device comprises an actuator to control adjustment of the size of at least part of the opening device.

The invention also provides in a further aspect an apparatus for inserting a surgical device at least partially through a wound opening, the apparatus comprising:—

a conveying device insertable at least partially through a wound opening to convey a surgical device at least partially through the wound opening; and
an opening device of the invention.

According to another aspect of the invention there is provided an apparatus for inserting a surgical device at least partially through a wound opening, the apparatus comprising:—

a conveying device insertable at least partially through a wound opening to convey a surgical device at least partially through the wound opening; and
a handle for gripping the apparatus; the handle being radially offset from the longitudinal axis of the conveying device.

In one embodiment of the invention the conveying device has a lumen extending at least partially therethrough, through which a laparoscope may extend. Because the handle is radially offset, this arrangement facilitates ease of passage of the laparoscope through the lumen. The lumen may extend parallel to the longitudinal axis of the conveying device. The lumen may extend in-line with the longitudinal axis of the conveying device. The apparatus may be asymmetrical about the longitudinal axis of the conveying device.

The invention also provides in a further aspect a method of inserting a surgical device at least partially through a wound opening, the method comprising the steps of: —

providing a surgical device;
creating a wound opening;
holding at least part of the surgical device in a low-profile configuration; and
conveying the surgical device at least partially through the wound opening.

In one embodiment of the invention the surgical device is conveyed at least partially through the wound opening using a conveying device, and the surgical device is held in the low-profile configuration using a holder element. The holder element may be disassembled from the conveying device. The holder element may be disassembled from the surgical device.

In one embodiment of the invention the method comprises the step of releasing the surgical device. The method may comprise the step of collapsing at least part of the surgical device to the low-profile configuration before conveying at least partially through the wound opening. The holder element may remain externally of the wound opening.

### BRIEF DESCRIPTION OF THE DRAWINGS

The invention will be more clearly understood from the following description of some embodiments thereof, given by way of example only, with reference to the accompanying drawings, in which:—

FIG. 1 is an isometric view of a conveying device of an apparatus according to the invention;

FIG. 2 is an enlarged, isometric view of a part of the conveying device of FIG. 1;

FIG. 3 is an isometric view of the apparatus according to the invention and a surgical device;

FIGS. 4 to 7 are isometric views of the apparatus and the surgical device of FIG. 3, in use;

FIGS. 8 and 9 are cross-sectional, side views of the surgical device of FIG. 3, in use;

FIGS. 10 and 11 are isometric views of the apparatus and the surgical device of FIG. 3, in use;

FIG. 12 is an isometric view of another apparatus according to the invention and a surgical device;

FIG. 13 is an isometric view of the apparatus and the surgical device of FIG. 12, in use;

FIG. 14 is an isometric view of a further apparatus according to the invention and a surgical device;

FIGS. 15 and 16 are isometric views of the apparatus and the surgical device of FIG. 14, in use;

FIG. 16(a) is an isometric view of a holder element of another apparatus according to the invention;

FIG. 16(b) is a plan view of the holder element of FIG. 16(a);

FIGS. 16(c) and 16(d) are isometric views of the apparatus according to the invention and a surgical device, in use;

FIG. 16(d)(i) is a plan view from above of the apparatus and the surgical device of FIGS. 16(c) and 16(d) in a storage configuration in a pack;

FIG. 16(d)(ii) is a plan view from below of the apparatus and the surgical device of FIG. 16(d)(i) in the storage configuration in the pack;

FIG. 16(d)(iii) is a plan view from above of the apparatus and the surgical device of FIG. 16(d)(i) in a holding configuration in the pack;

FIG. 16(e) is an isometric view of a holder element of another apparatus according to the invention;

FIG. 16(f) is a side view of the holder element of FIG. 16(e);

FIG. 16(g) is a plan view of the holder element of FIG. 16(e);

FIG. 16(h) is an isometric view of a holder element of a further apparatus according to the invention and a surgical device;

FIGS. 16(i) to 16(n) are isometric views of another apparatus according to the invention and a surgical device, in use;

FIG. 16(o) is an isometric view of a holder element of another apparatus according to the invention;

FIG. 16(p) is an isometric view of the holder element of FIG. 16(o) and a surgical device;

FIGS. 16(q) to 16(t) are isometric views of the apparatus according to the invention and the surgical device of FIG. 6(p), in use;

FIG. 17 is an isometric view of the conveying device of FIG. 1, in use;

FIG. 18 is a view similar to FIG. 1 of a conveying device of another apparatus according to the invention;

FIG. 19 is a side view of another apparatus according to the invention in a narrowed configuration;

FIG. 20 is an end view of the apparatus of FIG. 19 in the narrowed configuration;

FIGS. 21 and 22 are views similar to FIGS. 19 and 20 of the apparatus of FIG. 19 in a widened configuration;

FIGS. 23 to 25 are partially cross-sectional, side views of another apparatus according to the invention, in use;

FIG. 26 is an isometric view of another apparatus according to the invention and a surgical device; and

FIG. 27 is an end view of the apparatus and the surgical device of FIG. 26.

### DETAILED DESCRIPTION

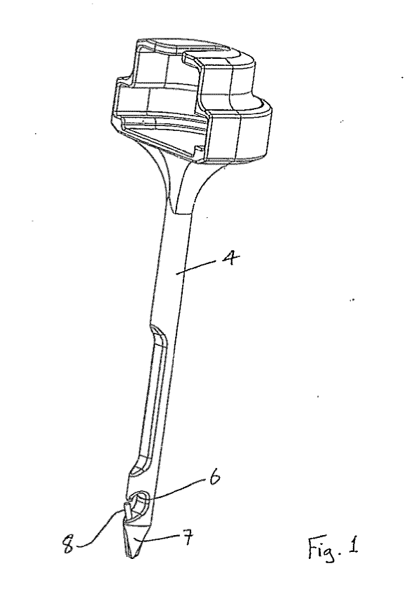

Referring to the drawings, and initially to FIGS. 1 to 11 thereof, there is illustrated an apparatus 1 according to the invention. The apparatus 1 is suitable for inserting a surgical device, such as a wound retractor device 2, at least partially through a wound opening 3. The apparatus 1 comprises a conveying device 4 and a separate holder element 5. The holder element 5 is formed separately from the conveying device 4.

In this case the wound retractor device 2 comprises a distal ring 52 for location internally of the wound opening 3 to anchor the wound retractor device 2 in position retracting the wound opening 3.

The conveying device 4 is insertable at least partially through the wound opening 3 to convey the wound retractor device 2 at least partially through the wound opening 3. The conveying device 4 comprises a hook element 6 for engaging the wound retractor device 2. The hook element 6 is non-circular in cross-section.

A bladeless tip 7 is provided mounted to the distal end of the conveying device 4 to create the wound opening 3 by forcing tissue apart. The tip 7 has a flattened, non-symmetrical shape in cross-section. In this case the major axis of the tip 7 is arranged at 90° to the major axis of the hook element 6.

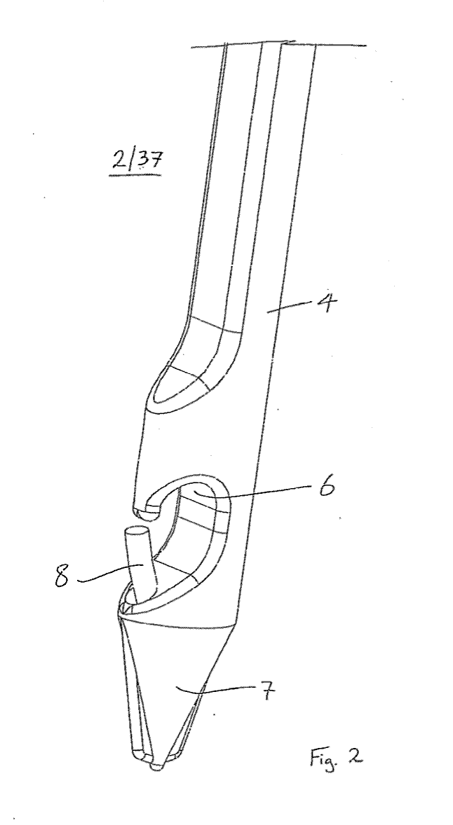

FIG. 1 illustrates the bladeless hook introducer 4 with a soft rubber skin deflector 8. The soft rubber skin deflector 8 prevents skin layers snagging on the hook 6 during introduction of the distal ring 52 of the wound retractor device 2 (FIG. 2).

The holder element 5 is provided in this case in the form of an annular flange arranged concentrically around the conveying device 4. The holder element 5 extends radially outwardly from the conveying device 4. The holder element 5 may be employed to hold at least part of the wound retractor device 2 in a low-profile configuration.

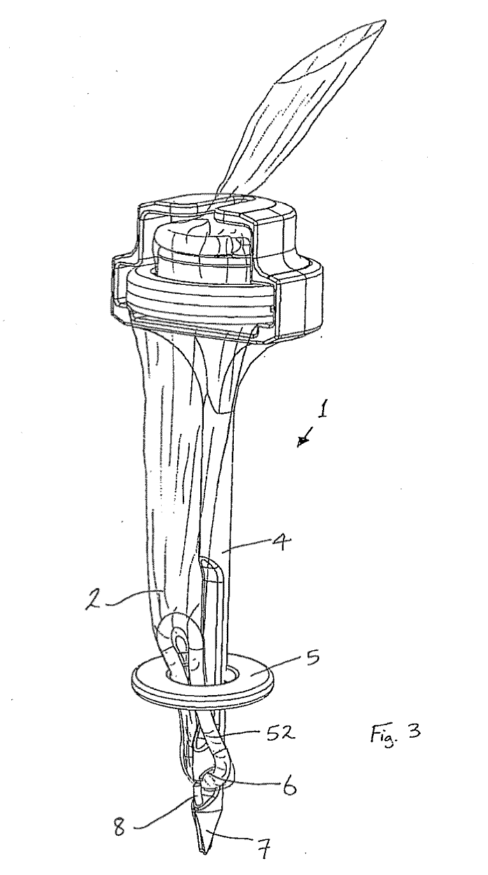

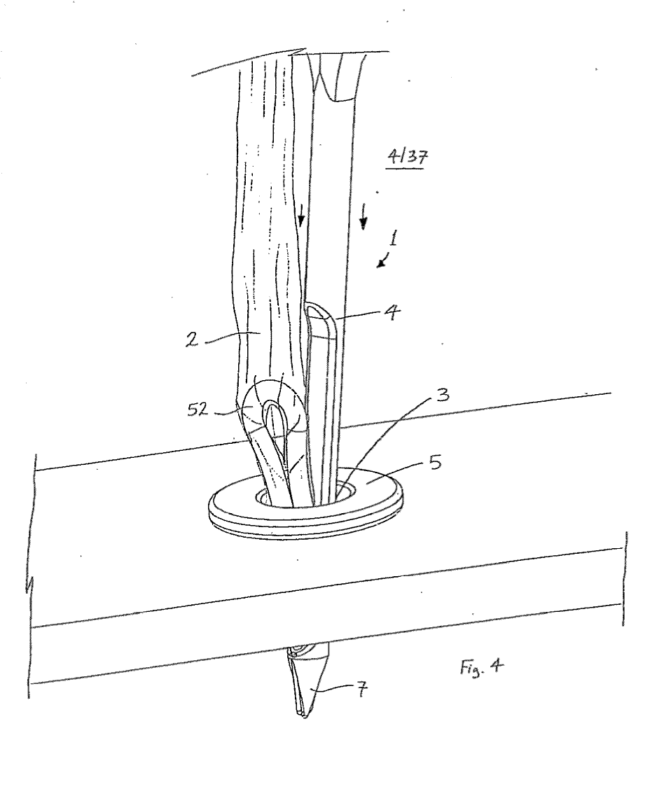

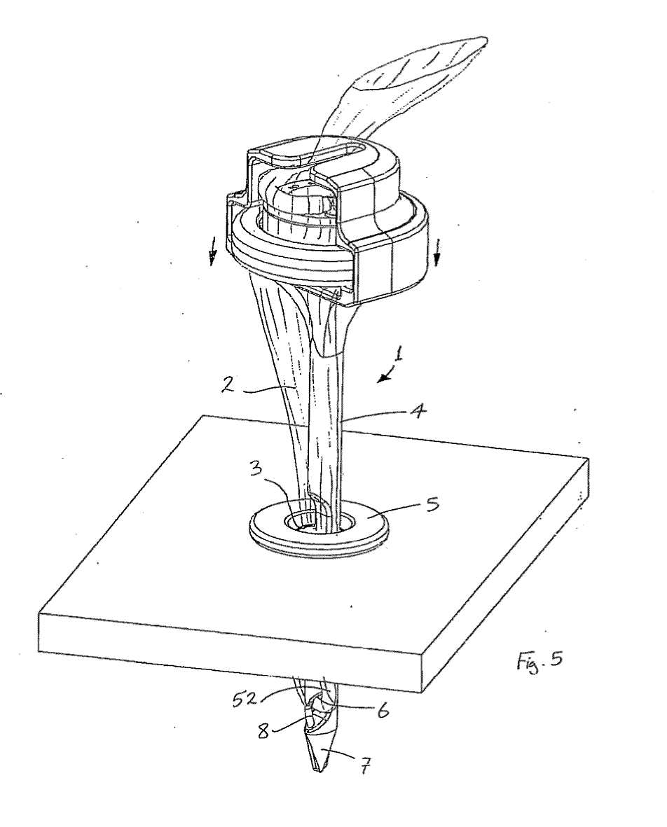

The holder element 5 is slidable relative to the conveying device 4 along the conveying device 4 between a holding configuration in which at least part of the wound retractor device 2 is held in the low-profile configuration (FIG. 3), and a release configuration in which the wound retractor device 2 is released (FIGS. 5 and 6). As the conveying device 4 is inserted through the wound opening 3, the holder element 5 engages with the external surface of the tissue (FIG. 4). In this manner the holder element 5 is moved from the holding configuration to the release configuration. The holder element 5 remains detached from the conveying device 4 in both the holding configuration and in the release configuration.

The holder element 5 may be disassembled from the conveying device 4 after the wound retractor device 2 has been conveyed through the wound opening 3. The conveying device 4 may be withdrawn through the central opening of the annular holder element 5.

After insertion of the wound retractor device 2, the conveying device 4 is removed from the wound opening 3, and the holder element 5 remains in position externally of the wound opening 3 (FIG. 8).

In FIG. 5, the ring 52 has been compressed passing through the compression washer 5 which results in more streamlined entry.

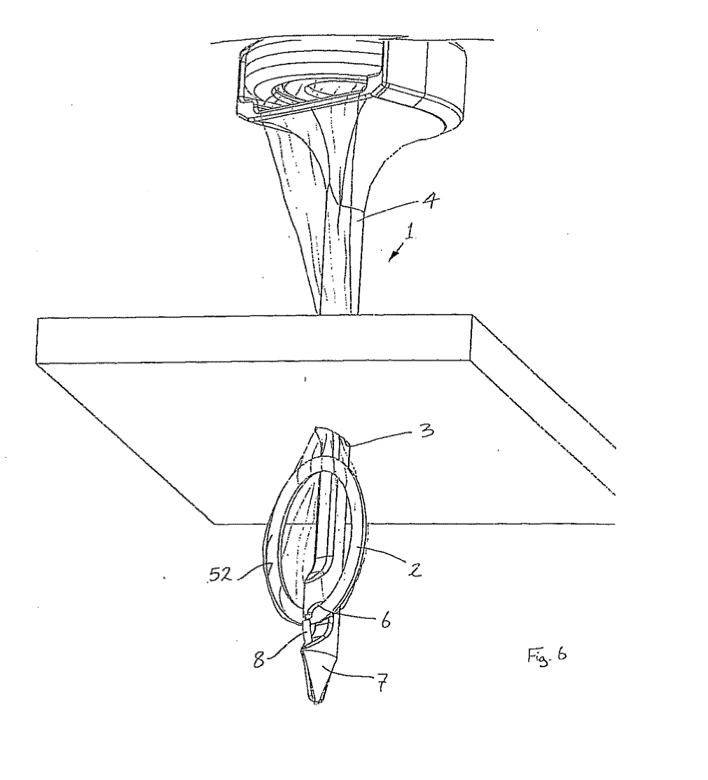

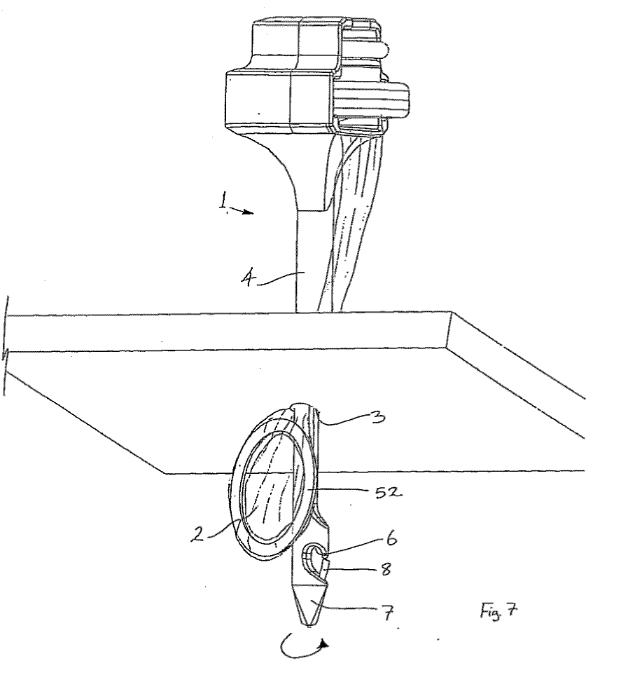

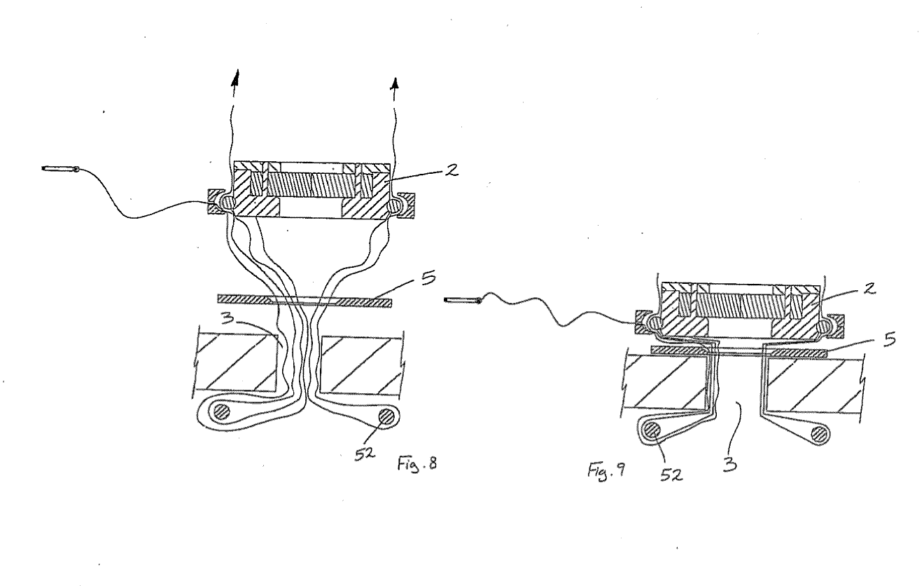

In FIG. 7, in order to release the distal ring 52 from the hook 6 and the soft rubber skin deflector 8, the user rotates the bladeless hook introducer 4 within the incision 3. The soft rubber skin deflector 8 deforms easily to release the distal ring 52. The user then removes the introducer 4.

FIG. 8 illustrates the compression washer 5, and the distal ring 52 deployed.

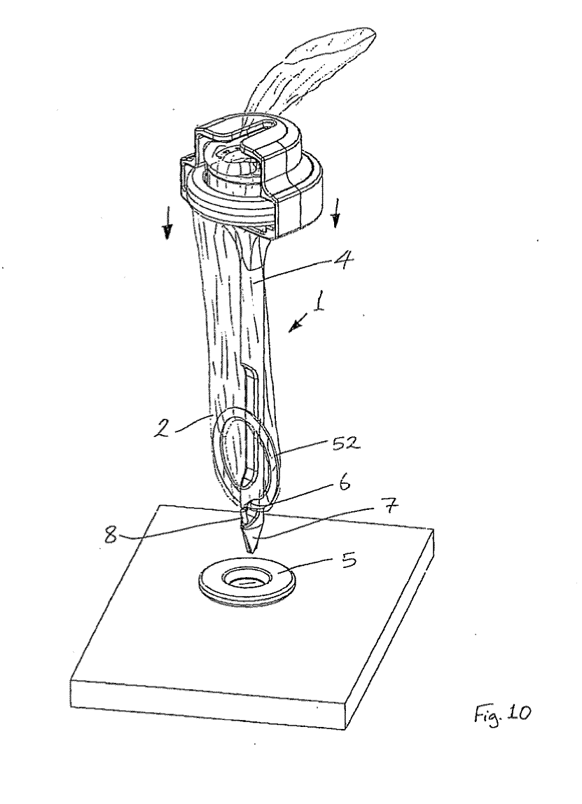

The holder element 5 may be arranged concentrically around the conveying device 4 before the conveying device 4 approaches the tissue. Alternatively the holder element 5 may be placed resting against the tissue before the conveying device 4 approaches the tissue (FIGS. 10 and 11). The conveying device 4 then passes through the central opening in the holder element 5 to compress the wound retractor device 2 to the low-profile configuration.

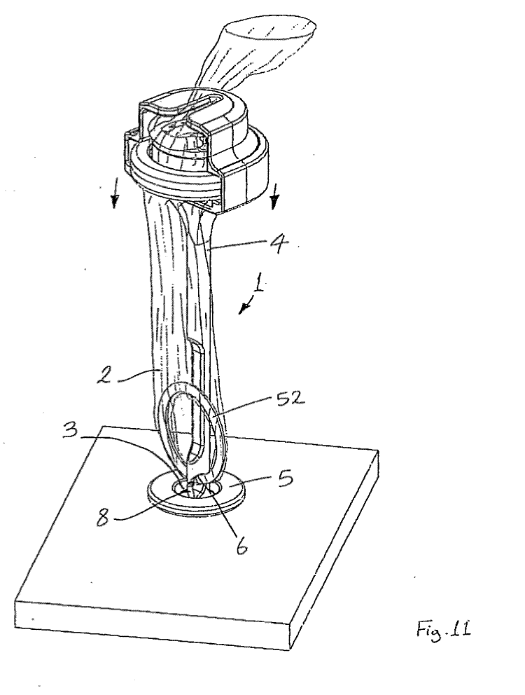

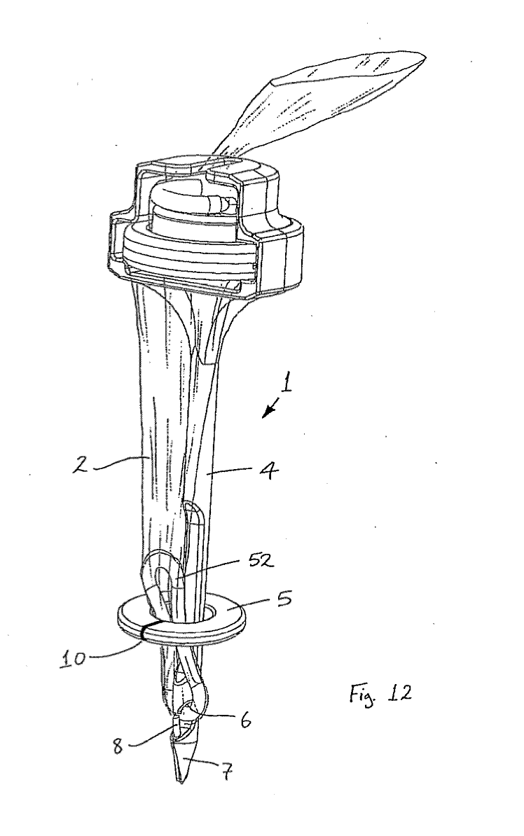

In the embodiment of FIGS. 12 and 13, a circumferential discontinuity 10 is provided in the holder element 5. In this case the circumferential discontinuity 10 is provided in the form of a cut through the annular flange 5. The discontinuity 10 enables the holder element 5 to be moved radially away from the conveying device 4 from the holding configuration (FIG. 12) to the release configuration (FIG. 13). In this manner the holder element 5 may be removed from being concentrically around the conveying device 4 in a fast, convenient manner.

The circumferential discontinuity 10 enables the holder element 5 to be disassembled from the wound retractor device 2 after the distal ring 52 of the wound retractor device 2 has been inserted through the wound opening 3.

In FIG. 12 the compression washer 5 has the split 10 in it to facilitate removal from around the introducer shaft 4.

FIG. 13 illustrates the compression washer 5 removed as a result of the split 10, and the distal ring 52 inside the abdomen.

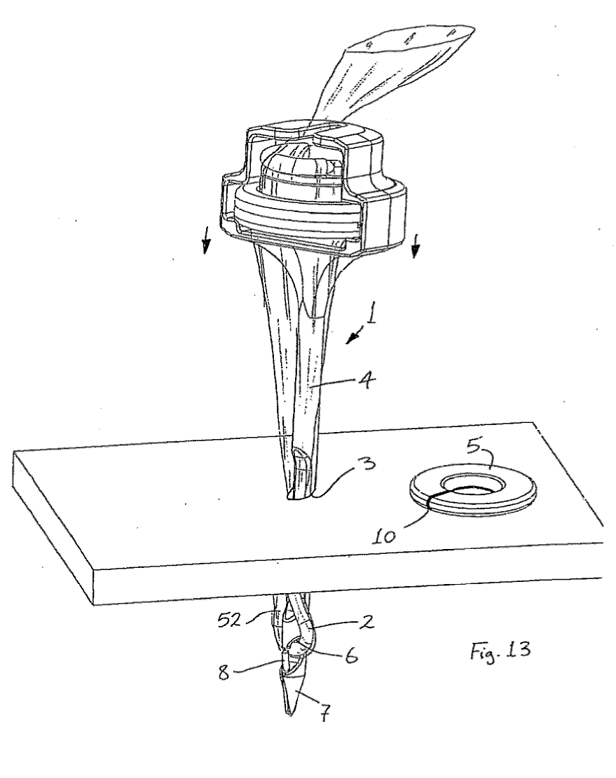

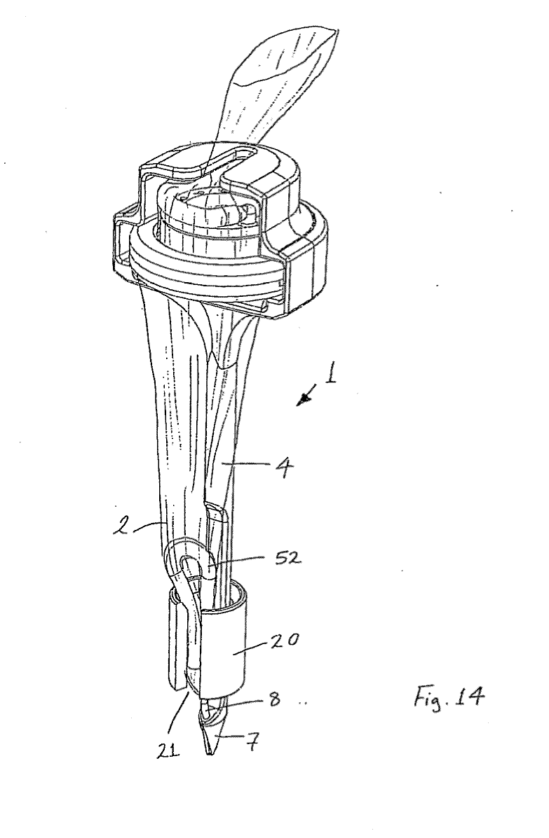

In the embodiment of FIGS. 14 to 16 the holder element 20 is provided in the form of a tubular element. The tubular element 20 has a circumferential discontinuity 21 in the form of an opening extending along the tubular element 20.

FIG. 14 illustrates the compression clip 20 with the gap 21 for removal. The clip 20 may be preloaded. The clip 20 may be hinged.

FIG. 15 illustrates an alternative in which the compression clip 20 is left on the abdomen and the introducer 4 is passed through it, compressing the distal ring 52.

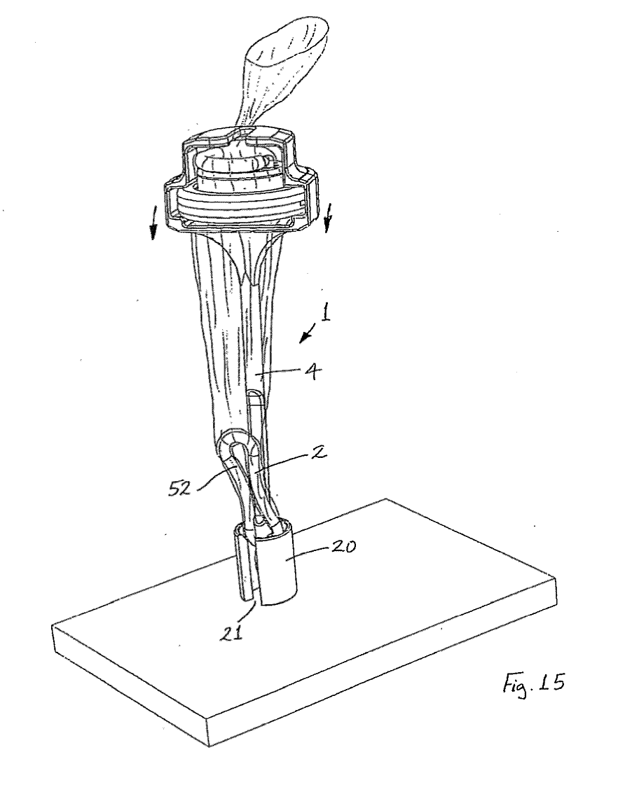

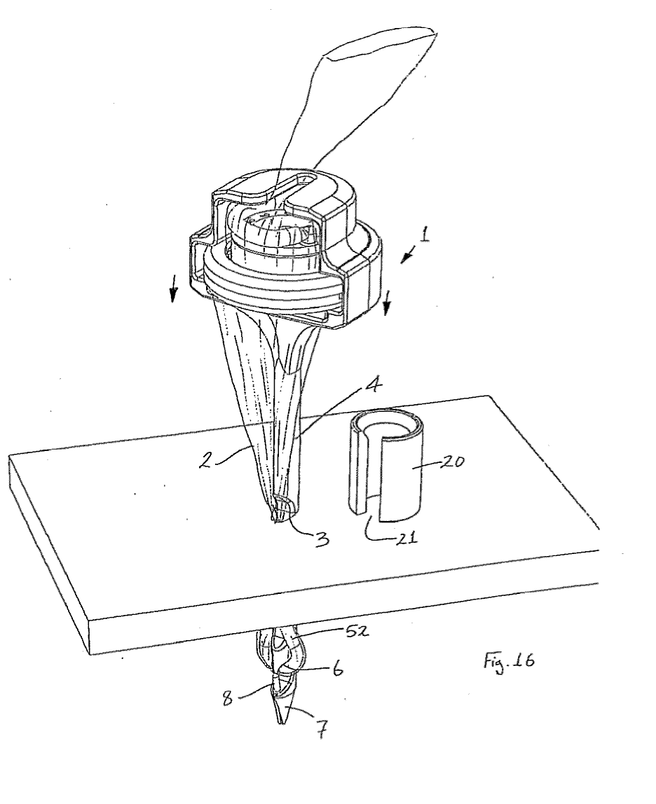

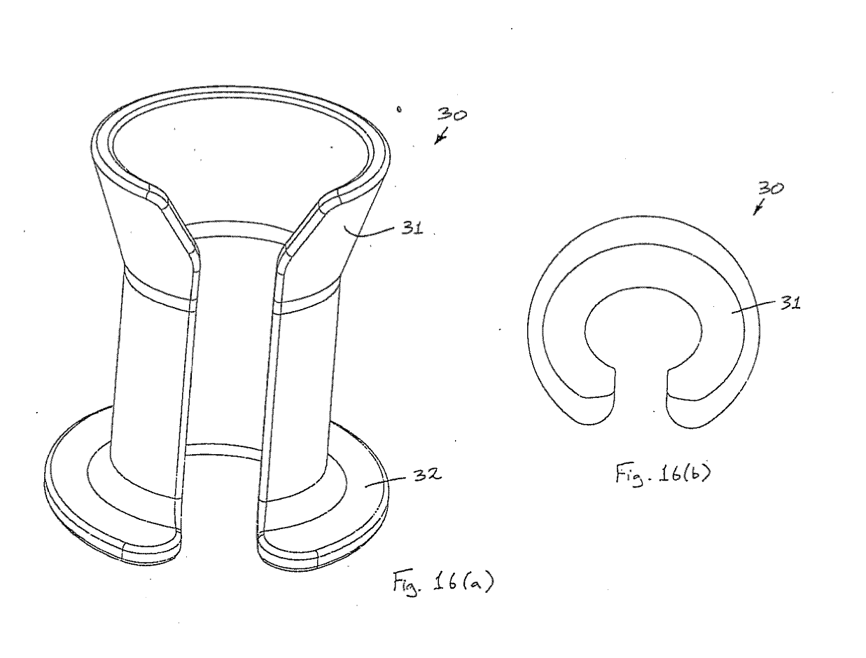

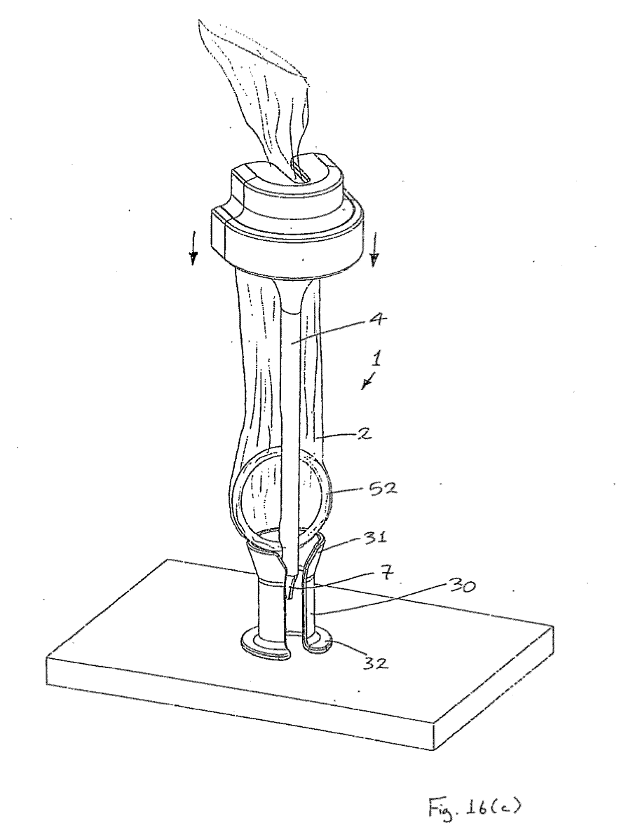

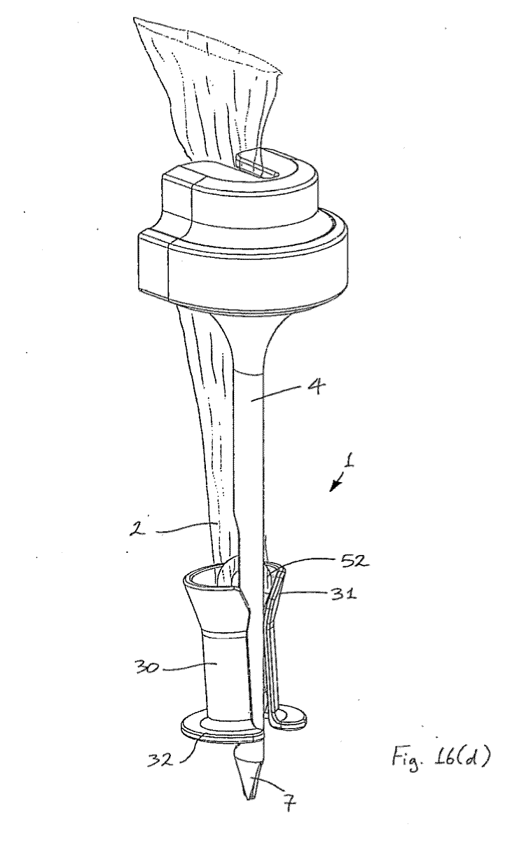

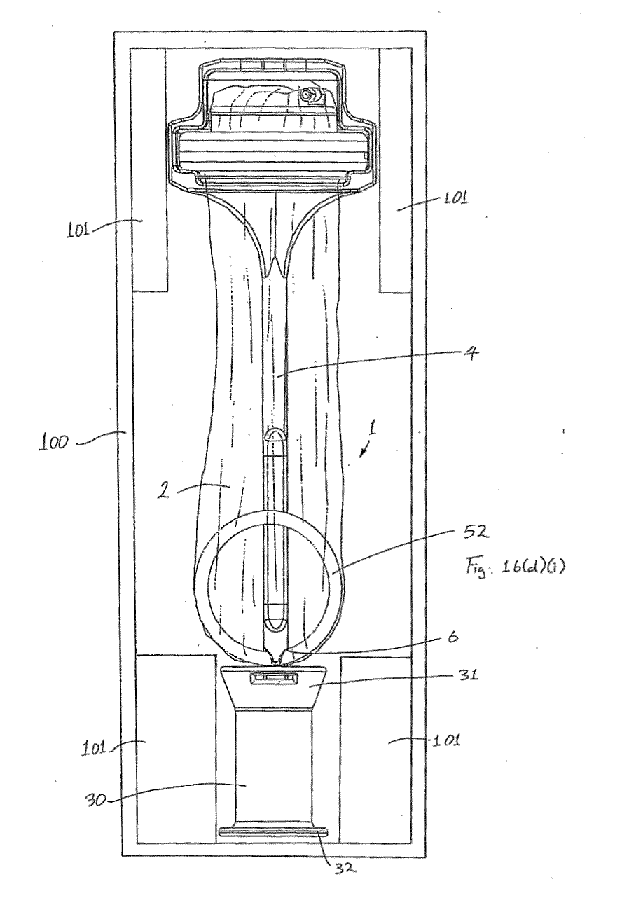

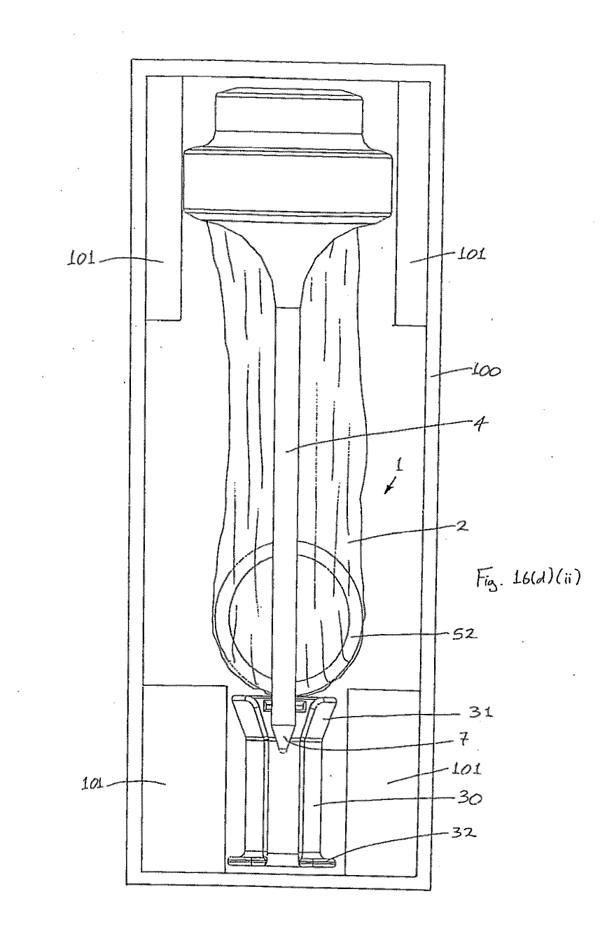

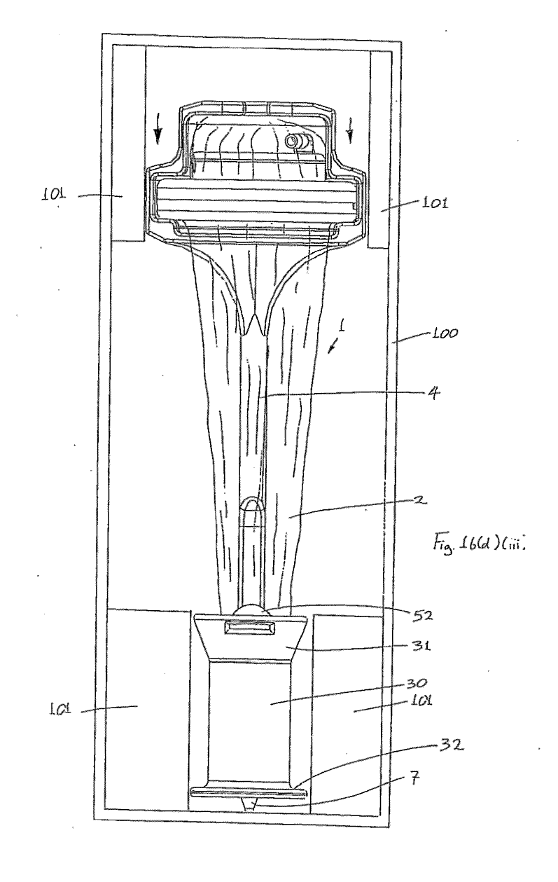

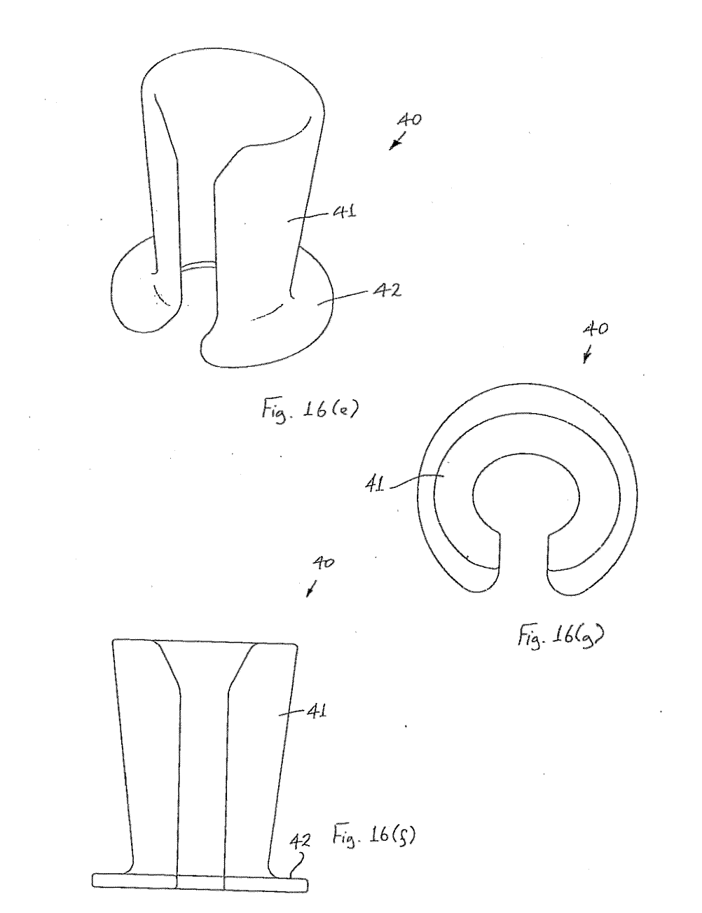

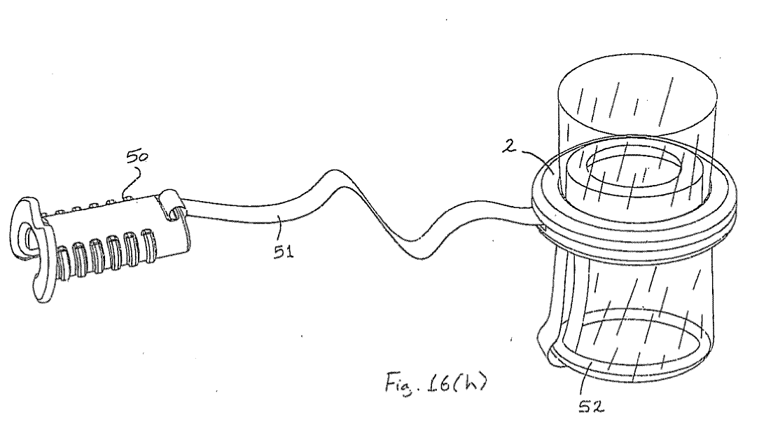

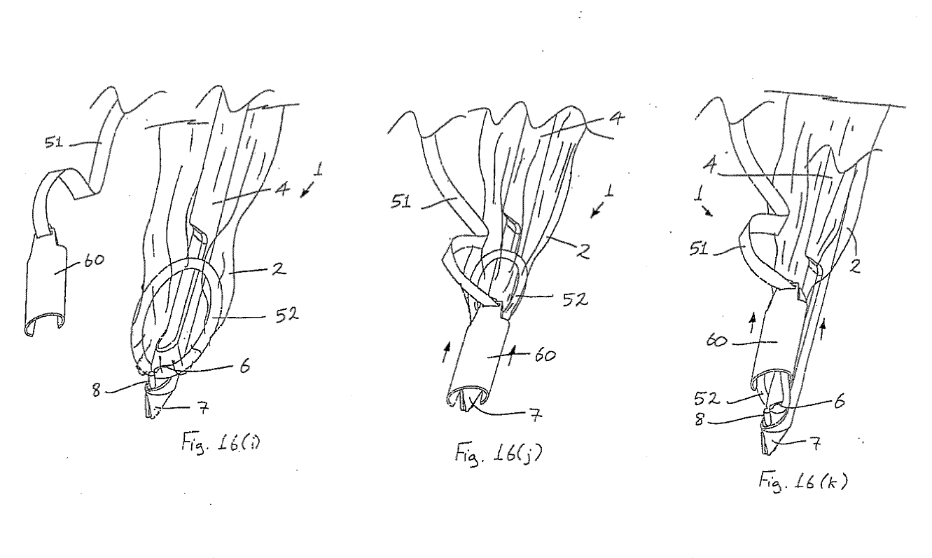

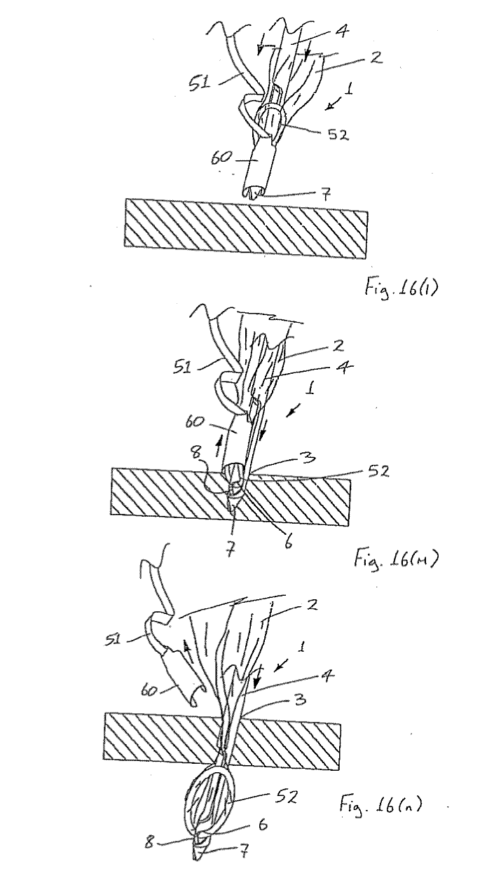

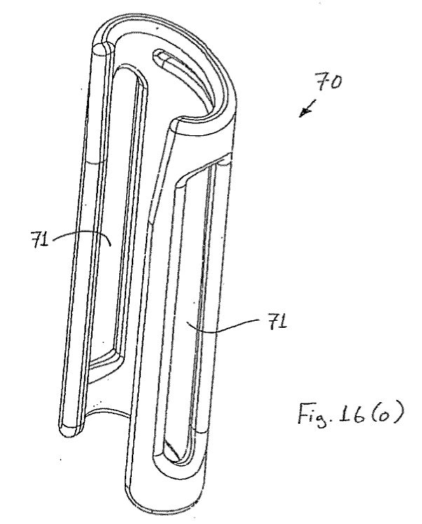

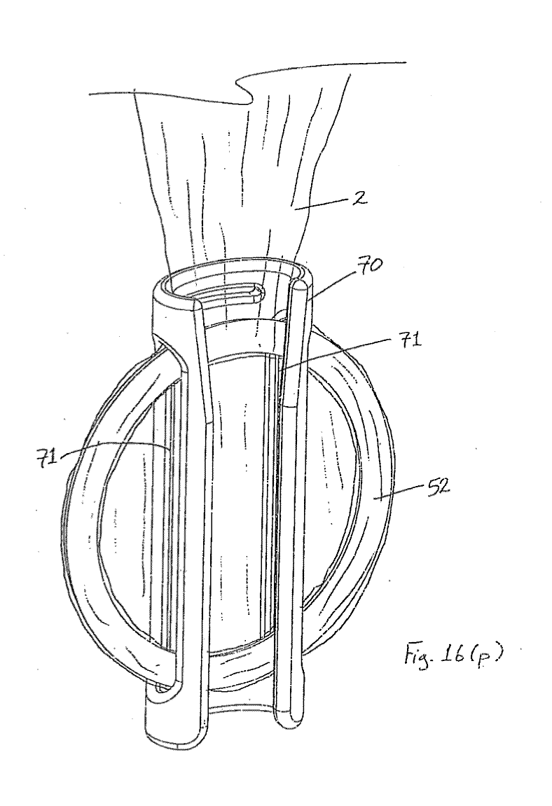

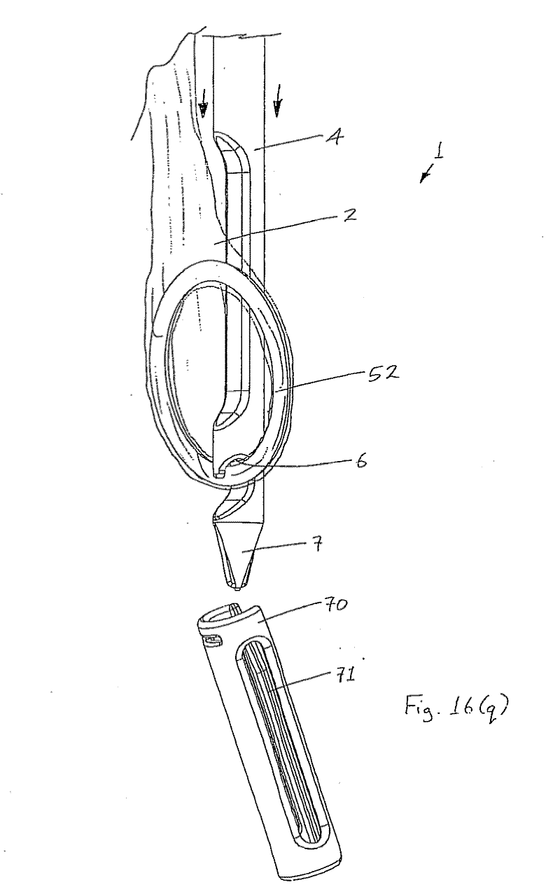

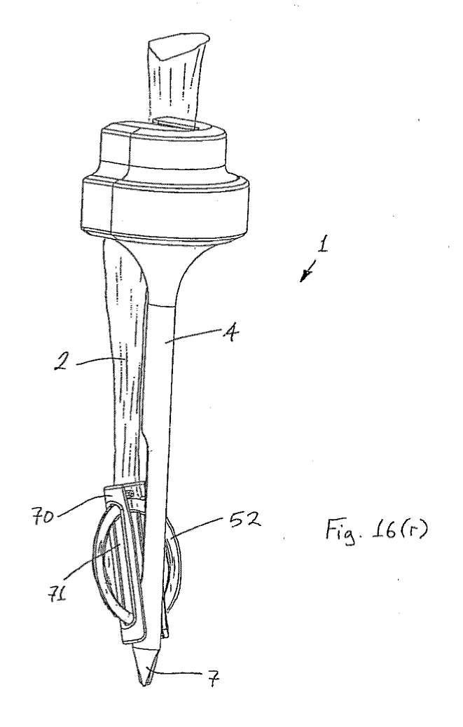

FIG. 16 illustrates that the split 21 allows the compression clip 20 to be removed once the distal ring 52 has passed through the incision 3.

In the embodiment of FIGS. 16(a) to 16(d) the holder element 30 is provided in the form of a tubular element. The tubular element 30 has a proximal funnel part 31 which tapers radially inwardly distally. The funnel part 31 assists collapse of the wound retractor device 2 from the release configuration to the low-profile configuration (FIGS. 16(c) and 16(d)).

The tubular element 30 has a distal flange part 32.

As illustrated in FIG. 16(b), the tubular element 30 has an elliptical shape in cross-section.

FIG. 16(a) illustrates the tapered lead-in 31 to promote compression of the distal ring 52, and the flange 32 for stability. FIG. 16(b) illustrates the elliptical cross-section.

FIG. 16(c) illustrates the clip 30 placed on a table top, and the introducer 4 and the distal ring 52 being pushed down into the clip 30 to load for the surgeon.

FIG. 16(d) illustrates the distal ring 52 streamlined by the clip 30. The surgeon can then pass the introducer 4 and the ring 52 easily through the abdominal wall.

In the embodiment of FIGS. 16(d)(i) to 16(d)(iii), the apparatus 1 and the wound retractor device 2 are housed in a pack 100. The conveying device 4 is slidable in the pack 100 relative to the tubular element 30 from a storage configuration in which the distal ring 52 of the wound retractor device 2 is released (FIGS. 16(d)(i) and 16(d)(ii)), to the holding configuration in which the distal ring 52 is held in the low-profile configuration (FIG. 16(d)(iii)).

FIG. 16(d)(i) illustrates the packaging 100 with packaging support 101, the clip 30, the wound retractor device 2, the hook introducer 4, and the distal ring 52. The wound retractor device 2 and the introducer 4 are in the packaging 100. The user pushes the introducer 4 down until the distal ring 52 is compressed in the clip 30.

FIG. 16(d)(ii) illustrates a rear view of the clip 30 with the funnel top 31.

In FIG. 16(d)(iii) the introducer 4 is pushed until the distal ring 52 is compressed in the clip 30. The funnel 31 aids compression.

In the embodiment of FIGS. 16(e) to 16(g) the holder element 40 is provided in the form of a tubular element. The tubular element 40 has a funnel part 41 which tapers radially inwardly distally. The funnel part 41 extends from the proximal end of the tubular element 40 to the distal flange part 42.

FIG. 16(f) illustrates that the taper 41 extends over the entire height of the streamlining clip 40.

In the embodiment of FIG. 16(h) the holder element 50 is provided in the form of a tubular element. The tubular element 50 is attached to the wound retractor device 2. In this case the tubular element 50 is attached to a release ribbon 51 of the wound retractor device 2. The release ribbon 51 may be employed to release the distal ring 52 of the wound retractor device 2 from a retracting configuration when it is desired to remove the distal ring 52 from the wound opening 3.

FIG. 16(h) illustrates the restraining sleeve 50, the ribbon 51, and the wound retractor device 2.

In the embodiment of FIGS. 16(i) to 16(n) the holder element 60 is provided in the form of a tubular element. In this case the tubular element 60 is not arranged concentrically around the conveying device 4 in the holding configuration. Instead the tubular element 60 is arranged radially offset from the conveying device 4 in the holding configuration (FIG. 16(k)). The tubular element 60 and the conveying device 4 are located in a side-by-side arrangement (FIG. 16(k)). The tubular element 60 is slidable relative to the conveying device 4 from the holding configuration to the release configuration (FIGS. 16(l) to 16(n)).

FIGS. 16(i) to 16(n) illustrate the restraining clip 60 for keeping the distal ‘O’-ring 52 compressed while being inserted.

FIG. 16(i) illustrates the restraining clip 60, the ribbon 51 attached to the clip 60, and the ‘O’ ring 52 on the hook 6. FIG. 16(j) illustrates the clip 60 sliding up over the ring 52 only. FIG. 16(k) illustrates the ring 52 compressed and the clip 60 sliding off.

FIG. 16(m) illustrates that as the introducer 4 is pushed into the skin, the clip 60 slides off FIG. 16(n) illustrates the clip 60 attached to the ribbon 51.

In the embodiment of FIGS. 16(o) to 16(t) the holder element 70 is provided in the form of a tubular element. The tubular element 70 has two openings 71 in the side walls of the tubular element 70 (FIG. 16(o)). Each opening 71 is provided in the form of an elongate slot.

The tubular element 70 is slidable relative to the conveying device 4 from a storage configuration in which the distal ring 52 of the wound retractor device 2 is released (FIG. 16(s)) to the holding configuration (FIG. 16(t)), and from the holding configuration (FIG. 16(t)) to the release configuration.

As illustrated in FIG. 16(p), in the storage configuration the tubular element 70 is arranged concentrically around the distal ring 52, with the distal ring 52 extending through the openings 71.

FIG. 16(o) illustrates the side slots 71 which allow the distal ring 52 to be pre-loaded in this streamlining clip 70. The slots 71 ensure that the ring 52 is not compressed. Thus the shelf-life is enhanced.

FIG. 16(p) illustrates the distal ring 52 loaded in the streamlining clip 70. The ring 52 is unrestrained due to the side slots 71.

FIG. 16(q) illustrates the distal ring 52 attached onto the hook 6. The streamlining clip 70 with the side slots 71 is then placed over the distal ring 52. This step may take place during manufacture. The surgeon would therefore not have to load the clip 70 onto the ring 52.

FIG. 16(r) illustrates the bladeless hook introducer 4, the streamlining clip 70 with the side slots 71 and the distal ring 52. FIG. 16(s) illustrates the base of the streamlining clip 70 engaging the skin, and the incision 3.

FIG. 16(t) illustrates that as the hook introducer 4 advances through the abdominal wall, the distal ring 52 becomes compressed by the streamlining clip 70. The ring 52 therefore passes through the wound opening 3 more easily.

It will be appreciated that there are a variety of different means for holding the wound retractor device 2 in the low-profile configuration. For example the user could simply squeeze the wound retractor device 2 into the low-profile configuration using the user's fingers (FIG. 17). FIG. 17 illustrates the distal ring 52 being compressed manually.

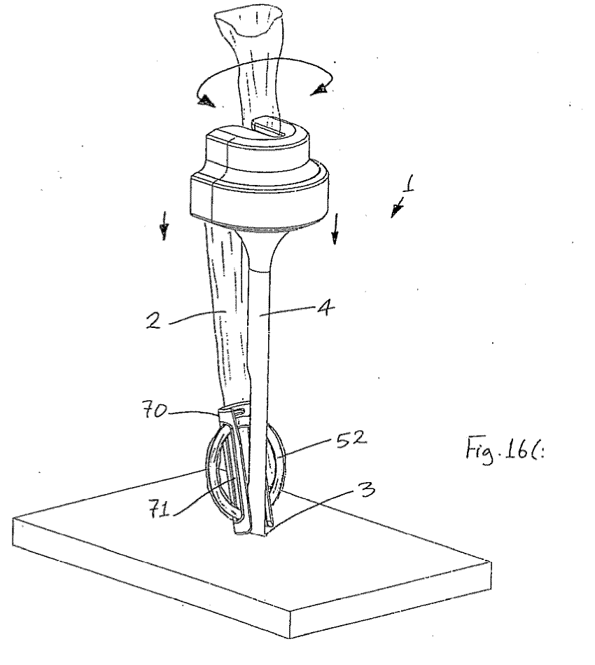

It will also be appreciated that the major axis of the tip 7 may be aligned with the major axis of the hook element 6, as illustrated in FIG. 18. FIG. 18 illustrates the alternative orientation of the cutting tip 7.

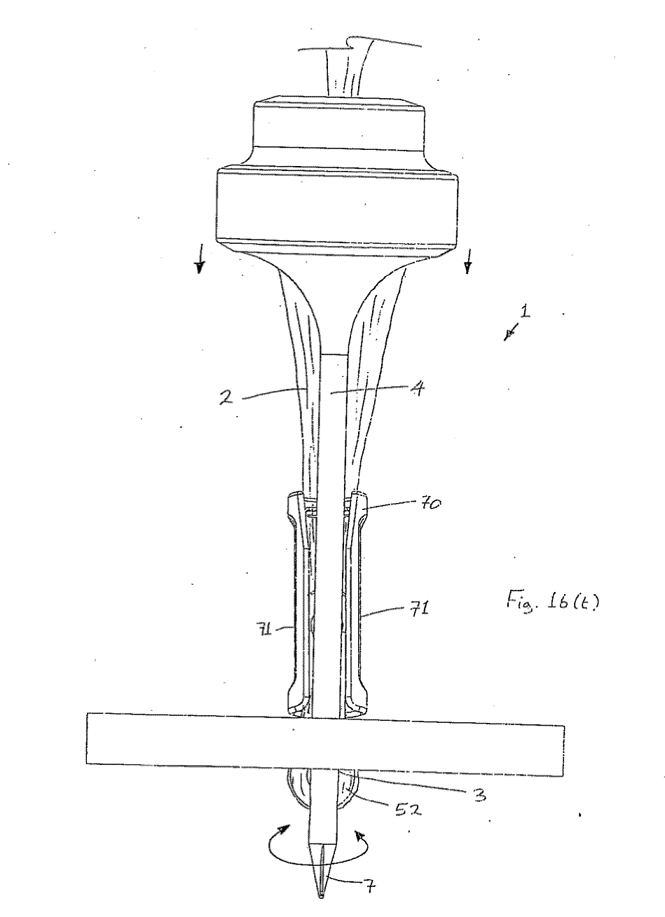

In the embodiment of FIGS. 19 to 22 the bladeless tip 80 is mounted to the distal end of the conveying device 4 to create the wound opening 3 by forcing tissue apart.

The tip 80 comprises a main body portion 81 and two side wing elements 82. Each of the wing elements 82 is movable relative to the main body portion 81 between a narrowed configuration (FIG. 19) and a widened configuration (FIG. 21). In this manner the width of the distal tip 80 may be adjusted. By adjusting the width of the distal tip 80, the size of the wound opening 3 created may be varied to suit particular requirements.

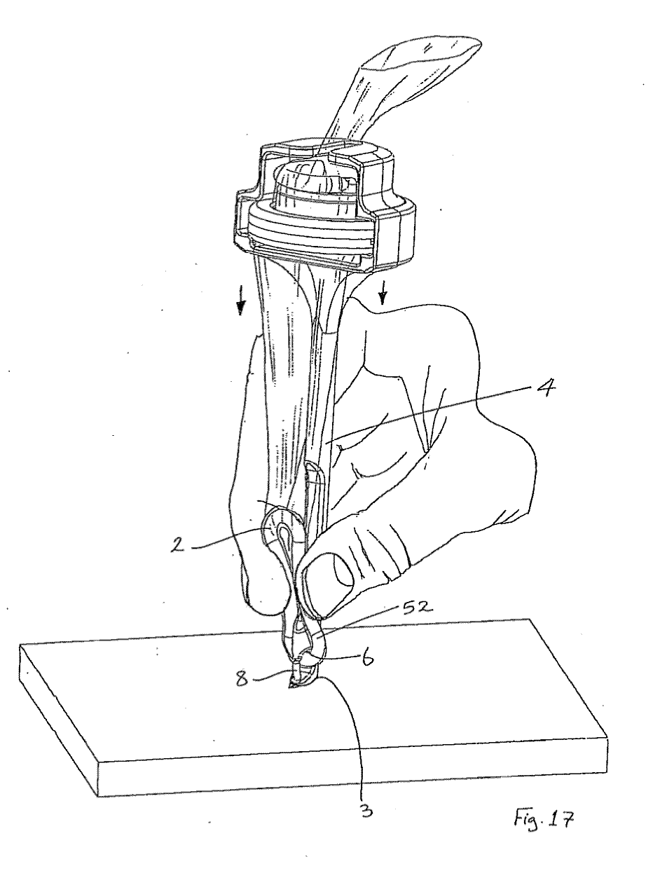

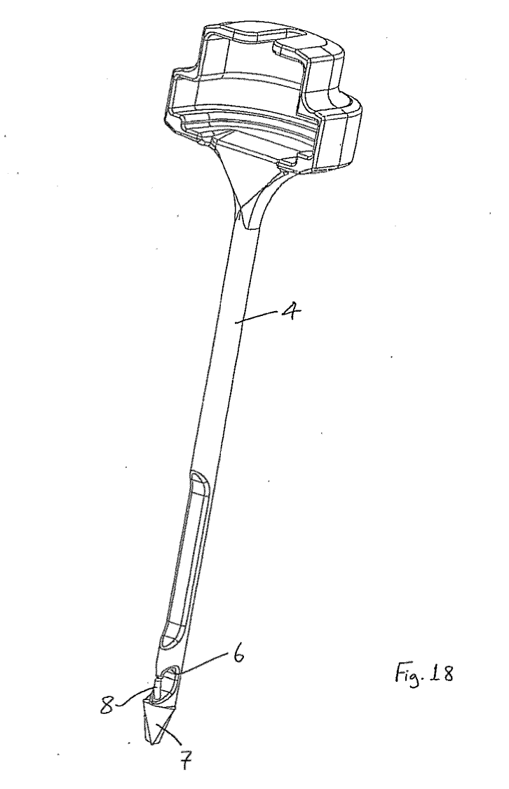

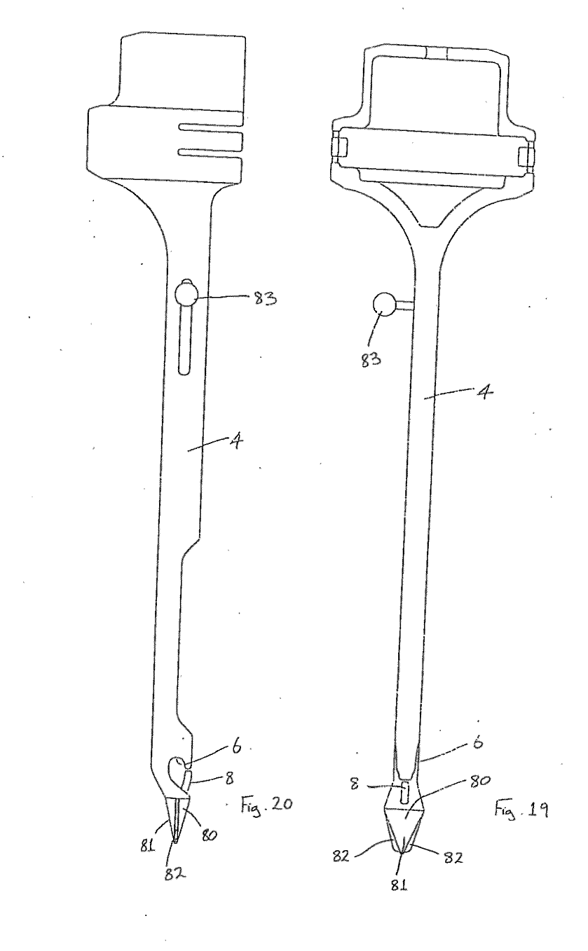

The tip 80 has a flattened, non-symmetrical shape in cross-section (FIGS. 20 and 22). The wing elements 82 are movable relative to the main body portion 81 in a direction parallel to the major axis of the tip 80.

An actuator handle 83 is provided for user control of adjustment of the width of the tip 80.

FIGS. 19 and 20 illustrate the adjustable cutting wings 82, and the thumbswitch 83 for the adjustable cutting wings 82. FIGS. 21 and 22 illustrate the thumbswitch 83 down, and the adjustable cutting wings 82 fanned out to create a larger incision.

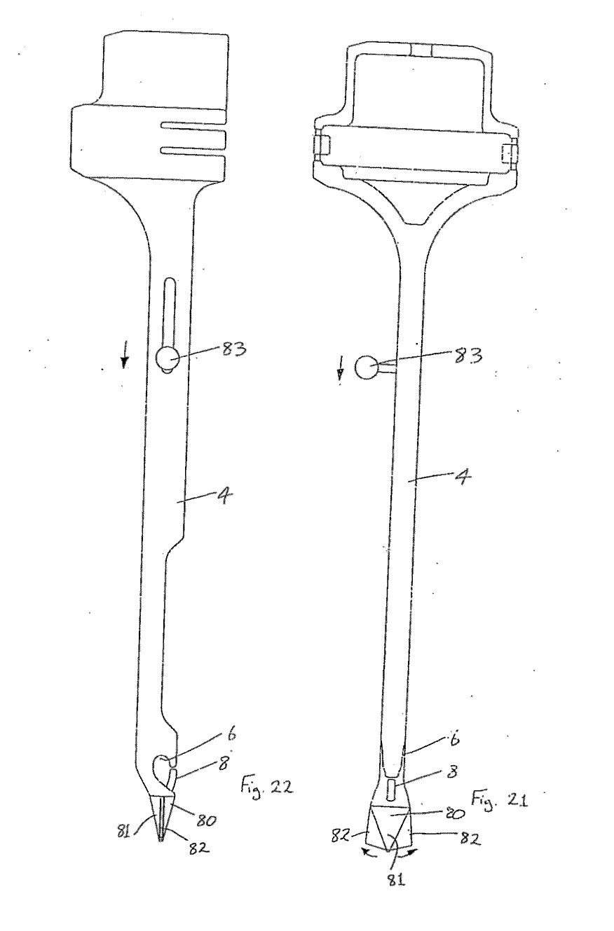

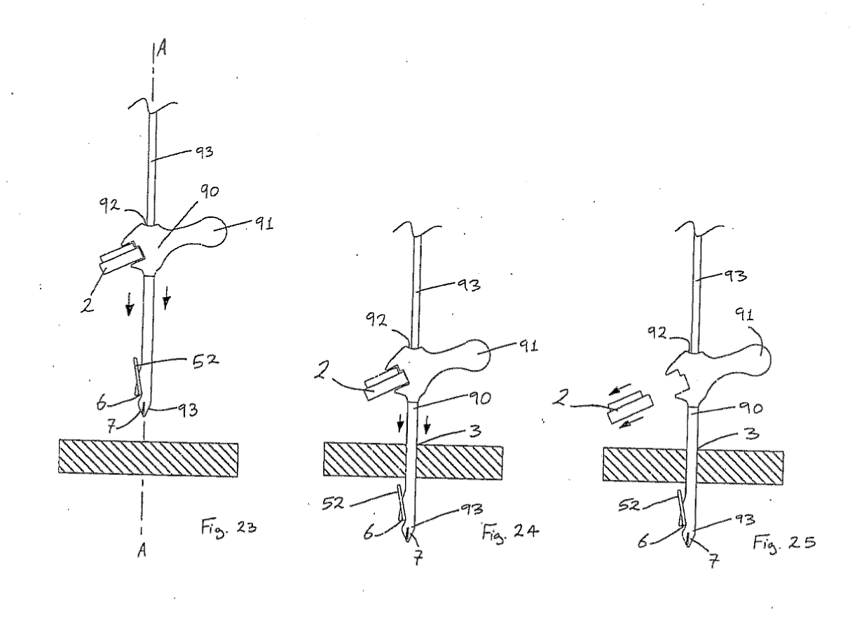

In the embodiment of FIGS. 23 to 25 the apparatus 90 comprises a handle 91 to facilitate a user gripping the apparatus 90.

A lumen 92 extends through the conveying device 4 from the proximal end of the conveying device 4 towards the distal tip 93. The lumen 92 extends parallel to and in-line with the longitudinal axis A-A of the conveying device 4. A laparoscope 93 may be extended through the lumen 92.

As illustrated in FIG. 23; the apparatus 90 is asymmetrical about the longitudinal axis A-A of the conveying device 4. In particular the handle 91 is radially offset from the longitudinal axis A-A of the conveying device 4. Because the handle 91 is radially offset from the longitudinal axis A-A, this provides for ease of passage of the laparoscope 93 through the lumen 92.

FIGS. 23 to 25 illustrate the handle 91 which allows more comfortable use with the laparoscope 93.

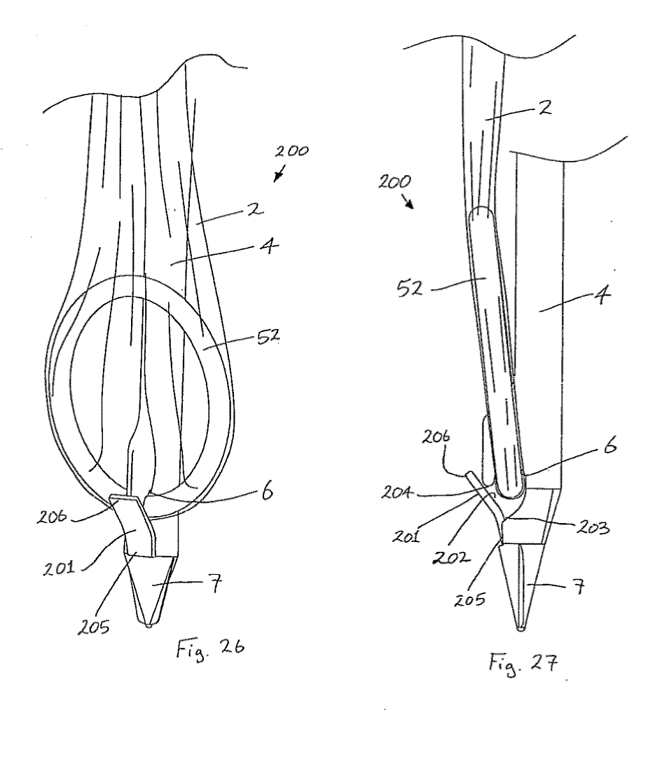

In FIGS. 26 and 27 there is illustrated another apparatus 200 according to the invention, which is similar to the apparatus of FIG. 18, and similar elements in FIGS. 26 and 27 are assigned the same reference numerals.

In this case the conveying device 4 comprises a resilient flap 201 which extends across the full width of a passageway 202 between the concave portion of the hook element 6 and externally of the conveying device 4. The passageway 202 facilitates location of the distal ring 52 in engagement with the hook element 6. She flap 201 may be used to selectively obstruct the full width of the passageway 202 to retain the distal ring 52 of the wound retractor device 2 in engagement with the hook element 6.

The flap 201 overlaps the passageway 202. In particular the distal end 205 of the flap 201 is located distally of the distal side 203 of the passageway 202, and the proximal end 206 of the flap 201 is located proximally of the proximal side 204 of the passageway 202.

The flap 201 is movable between an obstructing configuration in which the flap 201 extends across to obstruct the full width of the passageway 202 and a passage configuration in which the flap 201 is bent back to facilitate passage of the distal ring 52 of the wound retractor device 2 through the passageway 202 into and out of the concave portion of the hook element 6. In the obstructing configuration, the flap 201 retains the distal ring 52 in engagement with the hook element 6 during insertion of the wound retractor device 2 through the wound opening 3. The resilient nature of the flap 201 biases the flap 201 towards the obstructing configuration.

FIGS. 26 and 27 illustrate the hook introducer 200 with the hook protecting flap 201. FIG. 26 illustrates the retracting sleeve 2, the distal ring 52, the bladeless dissecting tip 7, and the flap 201 which covers the hook 6 to prevent skin layers getting snagged under the hook tip 204 or the ring 52. FIG. 27 illustrates the retracting sleeve 2, the distal ring 52, the shaft 4, the hook 6, and the flap 201 to cover the hook 6.

The invention is not limited to the embodiments hereinbefore described, with reference to the accompanying drawings, which may be varied in construction and detail.

1-73. (canceled)

74. An apparatus for inserting a surgical device at least partially through a wound opening, the apparatus comprising:—
a conveying device insertable at least partially through a wound opening to convey a surgical device at least partially through the wound opening; and
a holder element for holding at least part of the surgical device in a low-profile configuration.

75. An apparatus as claimed in claim 74 wherein the holder element is configured to be disassembled from the conveying device.

76. An apparatus as claimed in claim 75 wherein the holder element is configured to be disassembled from the conveying device after conveying of a surgical device at least partially through a wound opening.

77. An apparatus as claimed in claim 74 wherein the holder element is configured to be disassembled from a surgical device.

78. An apparatus as claimed in claim 74 wherein the holder element comprises a circumferential discontinuity.

79. An apparatus as claimed in claim 74 wherein the holder element is formed separately from the conveying device.

80. An apparatus as claimed in claim 74 wherein the holder element is movable relative to the conveying device between a holding configuration in which at least part of a surgical device is held in a low-profile configuration, and a release configuration in which the surgical device is released.

81. An apparatus as claimed in claim 80 wherein the holder element is slidable relative to the conveying device between the holding configuration and the release configuration.

82. An apparatus as claimed in claim 81 wherein the holder element is slidable relative to the conveying device along the conveying device.

83. An apparatus as claimed in claim 80 wherein the holder element is engagable with an external surface of tissue upon insertion of the conveying device at least partially through a wound opening to move the holder element from the holding configuration to the release configuration.

84. An apparatus as claimed in claim 80 wherein the holder element is movable radially relative to the conveying device between the holding configuration and the release configuration.

85. An apparatus as claimed in claim 74 wherein the holder element is configured to be arranged concentrically around the conveying device at least in the holding configuration.

86. An apparatus as claimed in claim 85 wherein the holder element is removable from being concentrically around the conveying device.

87. An apparatus as claimed in claim 74 wherein the holder element is configured to be arranged radially offset from the conveying device at least in the holding configuration.

88. An apparatus as claimed in claim 80 wherein the holder element is movable relative to the conveying device between a storage configuration in which at least part of a surgical device is released, and the holding configuration.

89. An apparatus as claimed in claim 88 wherein the holder element is slidable relative to the conveying device between the storage configuration and the holding configuration.

90. An apparatus as claimed in claim 88 wherein the holder element is configured to be arranged concentrically around at least part of a surgical device in the storage configuration.

91. An apparatus as claimed in claim 88 wherein the holder element comprises one or more openings through which part of a surgical device may extend in the storage configuration.

92. An apparatus as claimed in claim 74 wherein the holder element is configured to extend radially outwardly from the conveying device.

93. An apparatus as claimed in claim 74 wherein the holder element comprises an annular flange element.

94. An apparatus as claimed in claim 74 wherein the holder element comprises a tubular element.

95. An apparatus as claimed in claim 74 wherein the holder element is configured to assist collapse of at least part of a surgical device from a release configuration to a low-profile configuration.

96. An apparatus as claimed in claim 95 wherein at least part of the holder element comprises a funnel part.

97. An apparatus as claimed in claim 95 wherein at least part of the holder element is tapered.

98. An apparatus as claimed in claim 97 wherein at least part of the holder element tapers radially inwardly distally.

99. An apparatus as claimed in claim 74 wherein the holder element is non-circular in cross-section.

100. An apparatus as claimed in claim 99 wherein the holder element is elliptical in cross-section.

101. An apparatus as claimed in claim 74 wherein the holder element is configured to be attached to a surgical device.

102. An apparatus as claimed in claim 74 wherein the conveying device comprises a retaining element to retain a surgical device in engagement with the conveying device, and a passageway to facilitate location of the surgical device in engagement with the conveying device, the retaining element being configured to selectively obstruct the full width of the passageway.

103. An apparatus as claimed in claim 102 wherein the proximal end of the retaining element is located proximally of the proximal side of the passageway.

104. An apparatus as claimed in claim 102 wherein the distal end of the retaining element is located distally of the distal side of the passageway.

105. An apparatus as claimed in claim 102 wherein the retaining element overlaps the passageway.

106. An apparatus as claimed in claim 74 wherein the conveying device comprises a hook element to engage a surgical device.

107. An apparatus as claimed in claim 106 wherein the hook element is non-symmetrical in cross-section.

108. An apparatus as claimed in claim 106 wherein the hook element is non-circular in cross-section.

109. An apparatus as claimed in claim 74 wherein the apparatus comprises an opening device to create a wound opening.

110. An apparatus as claimed in claim 109 wherein the opening device is configured to create a wound opening by forcing tissue apart.

111. An apparatus as claimed in claim 109 wherein the opening device is mounted to the conveying device.

112. An apparatus as claimed in claim 109 wherein the opening device is non-symmetrical in cross-section.

113. An apparatus as claimed in claim 109 wherein the major axis of the opening device is substantially aligned with the major axis of the hook element.

114. A surgical assembly comprising:—
a surgical device; and
an apparatus as claimed in claim 74 for inserting the surgical device at least partially through a wound opening.

115. An opening device to create a wound opening, the size of at least part of the opening device being adjustable to vary the size of the wound opening created.

116. An apparatus for inserting a surgical device at least partially through a wound opening, the apparatus comprising:—
a conveying device insertable at least partially through a wound opening to convey a surgical device at least partially through the wound opening; and
an opening device as claimed in claim 115.

117. An apparatus for inserting a surgical device at least partially through a wound opening, the apparatus comprising:—
a conveying device insertable at least partially through a wound opening to convey a surgical device at least partially through the wound opening; and
a handle for gripping the apparatus;
the handle being radially offset from the longitudinal axis of the conveying device.

118) A method of inserting a surgical device at least partially through a wound opening, the method comprising the steps of:—
providing a surgical device;
creating a wound opening;
holding at least part of the surgical device in a low-profile configuration; and
conveying the surgical device at least partially through the wound opening.
